(19) United States
(12) Patent Application Publication
(10) Pub. No.: US 2003/0130701 A1
(43) Pub. Date: Jul. 10, 2003

(54) Opto-electric coupling device for photonic pacemakers and other opto-electric medical stimulation equipment

(76) Inventor: Victor Miller, Clarence, NY (US)

Correspondence Address: GREENWALD & BASCH, LLP, 349 WEST COMMERCIAL STREET, SUITE 2490, EAST ROCHESTER, NY, 14445, US

(21) Appl. No.: 10/037,720

(22) Filed: Jan. 4, 2002

(51) Int. Cl. A61N 1/36

(52) U.S. Cl. 607/9

(57) ABSTRACT

An opto-electric coupling device for photonic pacemakers and other opto-electric medical simulation equipment includes an opto-electrical transducer coupled to a pair of electrodes via a DC current discharge system that is adapted to counteract DC current flow caused by repeated application of electrical pulses to a body in which the electrodes are implanted.

### BACKGROUND OF THE INVENTION

1. Field of the Invention

The present invention relates to photonic pacemakers designed for compatibility with MRI diagnostic equipment, and to other opto-electric medical stimulation equipment, such as defibrillators, neural stimulators, and the like. More particularly, the invention concerns an opto-electric coupling device for use in delivering electrical pulses to implanted tissue while minimizing net DC current flow therein.

2. Description of Prior Art

By way of background, MRI compatible pacemakers for both implantable and wearable use have been disclosed in copending application Ser. Nos. 09/864,944 and 09,865,049, both filed on May 24, 2001, and copending Ser. Nos. 09/885,867 and 09/885,868, both filed on Jun. 20, 2001. In the aforementioned copending patent applications, whose contents are fully incorporated herein by this reference, the disclosed pacemakers feature photonic catheters carrying optical signals in lieu of metallic leads carrying electrical signals in order to avoid the dangers associated with MRI-generated electromagnetic fields. Electro-optical and opto-electrical transducers are used to convert between electrical and optical signals. In particular, a laser diode located in a main pacemaker enclosure is used to convert electrical pulse signals generated by a pulse generator into optical pulses. The optical pulses are carried over an optical conductor situated in a photonic catheter to a secondary housing, where they are converted by a photo diode array into electrical pulses for cardiac stimulation.

Despite the advances in pacemaker MRI compatibility offered by the devices of the above-referenced copending applications, there remains a problem of how to deliver stimulating electrical pulses to implanted body tissue, cardiac or otherwise, without building up a net DC current flow therein. Even minuscule amounts of net DC current will elicit chemical reactions that can deposit ever increasing amounts of unwanted chemical byproducts from the reactions. In a conventional pacemaker, the elimination of net DC current in implanted tissue can be achieved by inserting a capacitor in series with one of the pulse delivery electrodes. This arrangement is shown in FIG. 1, wherein a capacitor C1 is charged to battery potential between pulses through a resistor R1 and then quickly discharged via a transistor T1 into the cardiac tissue, thereby stimulating the heart. The fact that the capacitor C1, and only the capacitor C1, is in series with the heart insures that no net DC current can flow over a reasonable period of time (such as a few minutes). Between pulses, the distal side (electrode) of the capacitor C1 discharges to a little beyond zero and then goes back toward zero.

With a photonic pacemaker as contemplated by the above-referenced copending applications, the discharge pattern is not the same. If the output of the photo diode array is connected directly to the implanted tissue, the aforementioned DC current flow quickly builds up. If a capacitor is placed in series with the photo diode array, as is done in conventional non-photonic pacemakers, the capacitor starts with near zero volts on each side. The pulse from the photo diode array is negative and both sides of the capacitor instantly go negative by approximately the amount of the battery voltage. Then the photo diode array disconnects from the capacitor when it shuts off. Over several pulse cycles, the capacitor will quickly become fully charged, and will cease to permit DC current flow.

Accordingly, there is a challenge relative to the delivery of optically driven electrical stimulation signals to implanted tissue without residual DC current flow in the stimulated tissue over repeated cycles.

### SUMMARY OF THE INVENTION

The foregoing problems are solved and an advance in the art is provided by a novel opto-electric coupling device for use in photonic pacemakers and other opto-electric medical stimulation equipment. The coupling device includes an opto-electrical transducer adapted to generate periodic electrical pulses across a pair of electrodes adapted for implantation in a body. The coupling device further includes a DC current discharge system that counteracts DC current flow caused by application of the electrical pulses to a body in which the electrodes are implanted.

The DC current discharge system is connected to facilitate DC current discharge from the implanted body tissue. It is preferably implemented as an RC circuit using a capacitor and a resistor whose values are selected to provide an RC time constant in excess of a pulse width delivered by the opto-electrical transducer. The resistor of the RC circuit may be connected across the outputs of the opto-electrical transducer or it may be connected across the RC circuit's capacitor.

The invention further contemplates, respectively, a photonic pacemaker and an opto-electric medical stimulation system having the above-summarized opto-electric coupling device incorporated therein.

### BRIEF DESCRIPTION OF THE DRAWINGS

The foregoing and other features and advantages of the invention will be apparent from the following more particular description of preferred embodiments of the invention, as illustrated in the accompanying Drawing in which:

FIG. 1 is a schematic circuit view of a prior art electrical discharge system for preventing the build up of DC current flow in a conventional non-photonic pacemaker;

FIG. 2 is a block diagrammatic view of a photonic pacemaker;

FIG. 3 is a schematic circuit diagram showing an electrical pulse generator that may be used in the photonic pacemaker of FIG. 2;

FIG. 4 is a schematic circuit diagram showing an electrical pulse generator and voltage doubler that may be used in the photonic pacemaker of FIG. 2;

FIG. 5 is a schematic circuit diagram showing a first embodiment of an opto-electric coupling device in accordance with the invention;

FIGS. 6A and 6B are graphical illustrations of pulse waveforms that may be respectively input to and output from the opto-electric coupling device of the invention; and

FIG. 7 is a schematic circuit diagram showing a second embodiment of an opto-electric coupling device in accordance with the invention.

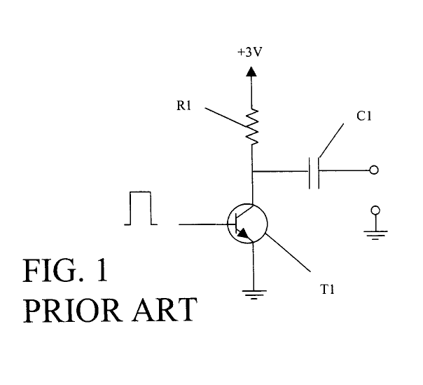

### DETAILED DESCRIPTION OF PREFERRED EMBODIMENTS

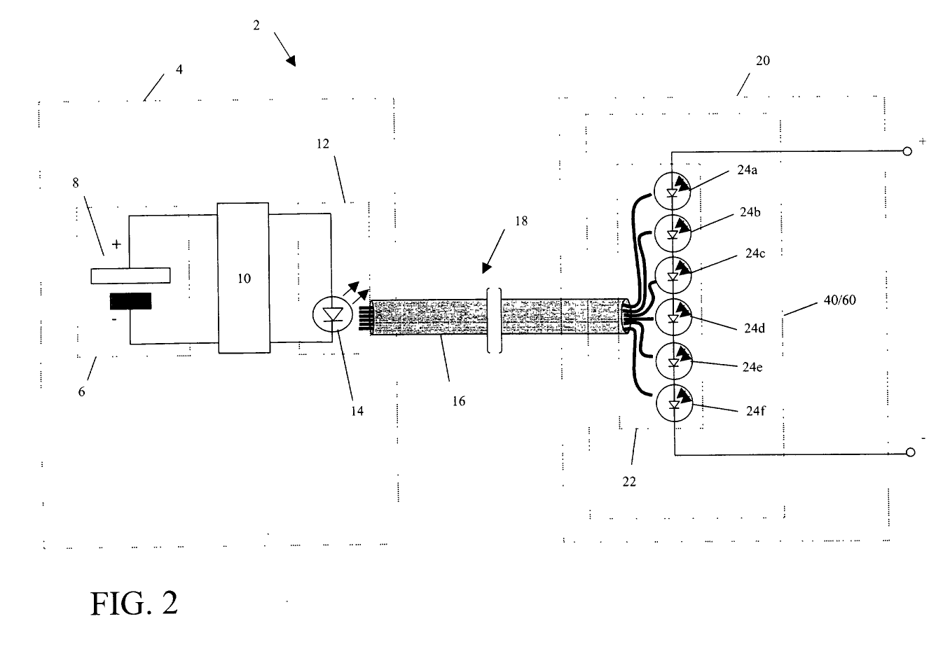

Turning now to FIG. 2, preferred embodiments of the invention will be described within the context of a photonic pacemaker 2. The pacemaker 2 comprises a main enclosure 4 that may either be implantable or wearable. The main enclosure 4 houses a power supply 6 that comprises one or more batteries 8. In particular, if the main enclosure 4 is designed for implantable use, a single battery 8 designed for implantable service could be used. Examples include conventional lithium iodine batteries (approximately 2.5-4.5 volts) and carbon monofloride batteries (approximately 1.5-3.5 volts). If the main enclosure is designed for external or wearable service, two or three conventional series-connected 1.5 volt batteries 8 could be used. In either case, the power supply 6 will typically provide a steady state DC output of at least about 3 volts.

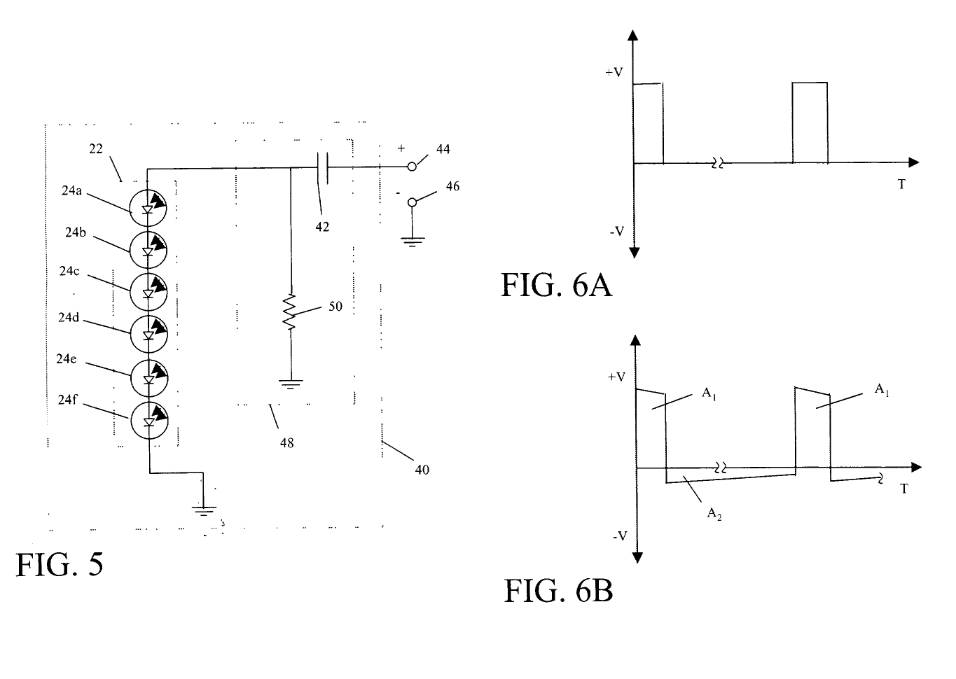

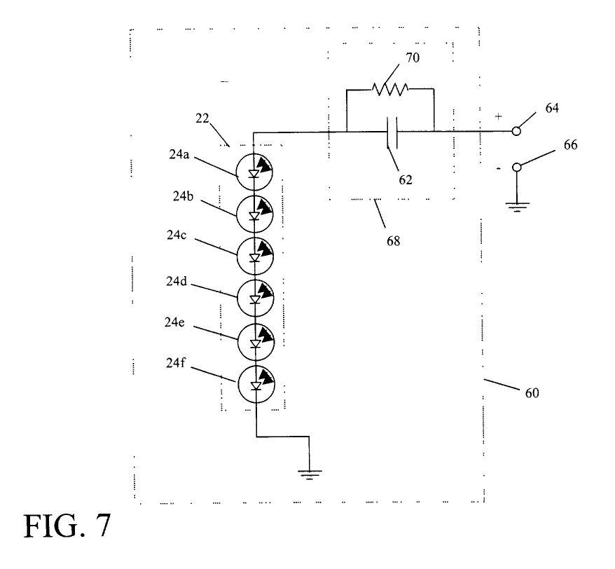

The power supply 6 powers an electrical pulse generator 10 (described in more detail below) that produces electrical pulses at its output. The electrical pulses drive the input of an electro-optical transducer 12, which is preferably implemented using a suitable laser light generator 14, such as a standard 150 milliwatt gallium arsenide laser diode. The electro-optical transducer 12 generates optical pulses at its output in correspondence with the electrical pulses output by the pulse generator 10. The optical pulses are applied to an optical conductor 16 (preferably a glass fiber optic element) situated in a photonic catheter 18. The photonic catheter 18 extends from the main enclosure 4 to a secondary enclosure 20. There, the optical conductor 16 terminates at an opto-electrical transducer 22. The opto-electrical transducer 22 may be constructed in a variety of ways, but is preferably implemented as an array of six series-connected photo diodes 24a-24f to develop the required photovoltaic output. The opto-electrical transducer 22 converts the light pulses into electrical pulses that are capable of stimulating the heart. As described in more detail below, the opto-electrical transducer 22 also forms part of an opto-electrical coupling device, embodiments of which are respectively shown in FIGS. 5 and 7 by way of reference numerals 40 and 60.

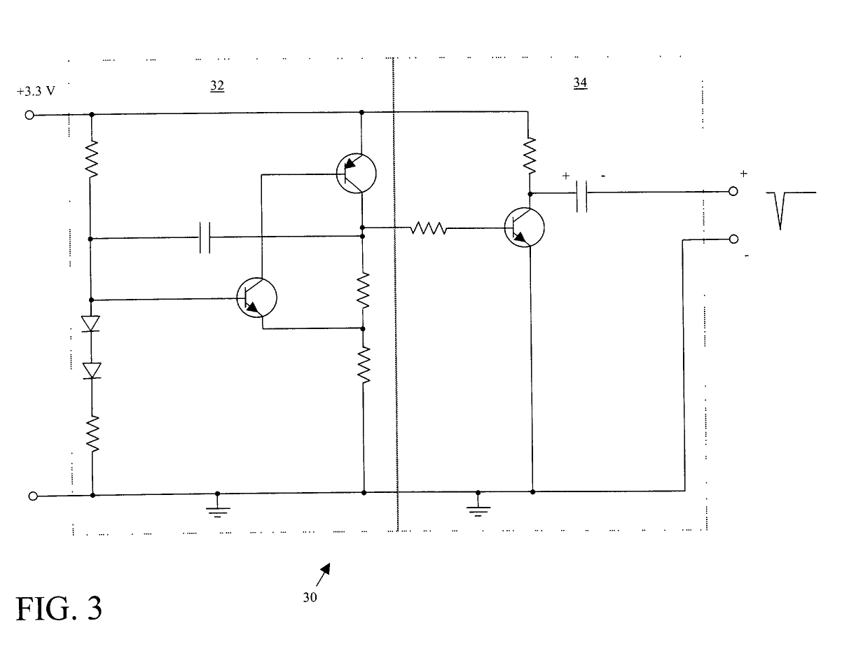

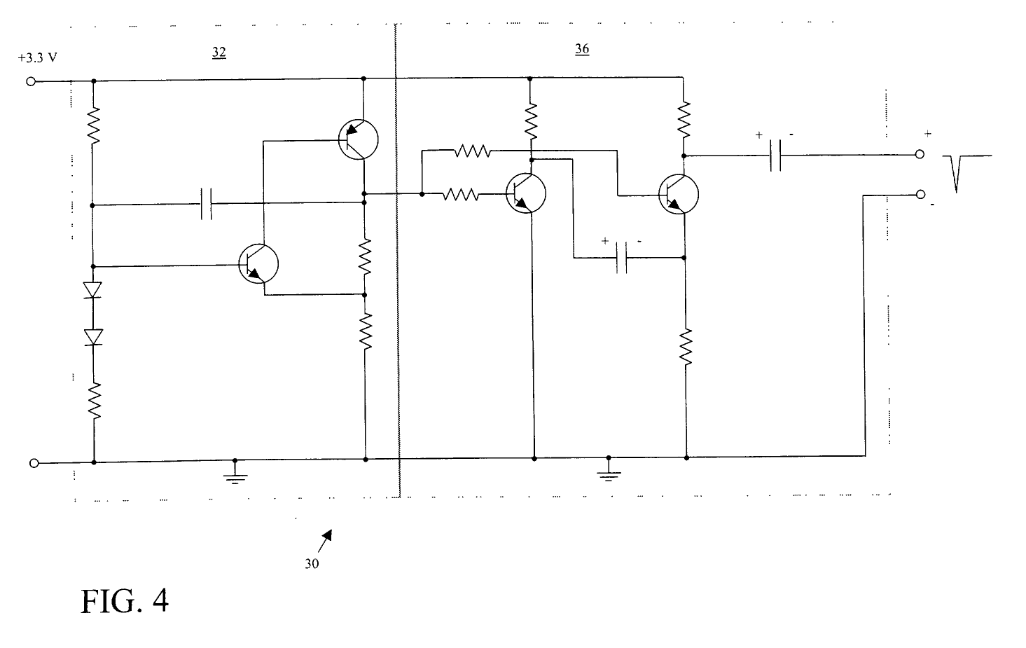

FIGS. 3 and 4 show two alternative circuit configurations that may be used to implement the pulse generator 10. Both alternatives are conventional in nature and do not constitute part of the present invention per se. They are presented herein as examples of the pulsing circuits that have been shown to function well in an implantable pacemaker environment. In FIG. 3, the pulse generator 30 includes an oscillator 32 and an amplifier 34. The oscillator 32 is a semiconductor pulsing circuit of the type disclosed in U.S. Pat. No. 3,508,167 of Russell, Jr. (the '167 patent). As described in the '167 patent, the contents of which are incorporated herein by this reference, the pulsing circuit forming the oscillator 32 provides a pulse width and pulse period that are relatively independent of load and supply voltage. The semiconductor elements are relegated to switching functions so that timing is substantially independent of transistor gain characteristics. In particular, a shunt circuit including a pair of diodes is connected so that timing capacitor charge and discharge currents flow through circuits that do not include the base-emitter junction of a timing transistor. Further circuit details are available in the '167 patent. The values of the components which make up the oscillator 32 can be selected to provide a conventional VOO pacemaker pulses varying from about 0.1-10 milliseconds duration at a period of about 1000 milliseconds.

The amplifier 34 of FIG. 3 is a circuit that uses a single switching transistor and a storage capacitor to deliver a negative-going pulse of approximately 3.3 volts across the pulse generator outputs when triggered by the oscillator 32. An example of such a circuit is disclosed in U.S. Pat. No. 4,050,004 of Greatbatch (the '004 patent), which discloses voltage multipliers having multiple stages constructed using the circuit of amplifier 34. As described in the '004 patent, the contents of which are incorporated herein by this reference, the circuit forming the amplifier 34 uses a 3.3 volt input voltage to charge a capacitor between oscillator pulses. When the oscillator 32 triggers, it drives the amplifier's switching transistor into conduction, which effectively grounds the positive side of the capacitor, causing it to discharge through the pulse generator's outputs. The values of the components which make up the amplifier 34 may be selected to produce an output potential of about 3.3 volts and a suitable current level for driving the electro-optical transducer 12.

The amplifier 36 of FIG. 4 is a circuit that uses a pair of the amplifier circuits of FIG. 3 to provide voltage doubling action. As described in the '004 patent, the capacitors are arranged to charge up in parallel between oscillator pulses. When the oscillator 32 triggers, it drives the amplifier's switching transistors into conduction, causing the capacitors to discharge in series to provide the required voltage doubling action. The values of the components that make up the amplifier 36 may be selected to produce an output potential of about 6.6 volts and a suitable current level for driving the electro-optical transducer 12.

Turning now to FIG. 5, a circuit diagram of a first exemplary opto-electric coupling device 40 is shown. The opto-electric coupling device includes the opto-electrical transducer 22, which is assumed to be illuminated by the photonic catheter 18 (see FIG. 2) for about 1 millisecond, and left dark for about 1000 milliseconds. When illuminated, opto-electrical transducer's photo diode array 24a-f will produce pulses of about 3 to 4 volts across its outputs. The positive side of the photo diode array 24a-f is connected via a high quality capacitor 42 to an implantable tip electrode (shown schematically at 44) that is adapted to be implanted in the endocardium of a patient. The negative side of the photo diode array 24a-f is connected to an implantable ring electrode (shown schematically at 46) that is adapted to be immersed in the blood of the patient's right ventricle. A DC current discharge system 48 comprising the capacitor 42 and a resistor 50 is used to attenuate DC current in the tissue implanted with the electrodes 44 and 46. In FIG. 5, the resistor 48 is connected across the outputs of the photo diode array 24a-f. The resistor 48 thus grounds one side of the capacitor 42 between pulses. The return path from the implanted tissue is the through the ring electrode 46.

The values of the capacitor 42 and the resistor 48 are selected so that the opto-electric coupling device 40 conveys a suitable stimulating signal to the electrodes 44 and 46, but in such a manner as to prevent any net DC current from flowing into the implanted tissue. A long RC time constant is desired so that the square waveform of the photo diode array output is delivered in substantially the same form to the implanted tissue. For a 1 millisecond pulse, the desired RC time constant should be substantially larger than 1 millisecond. By way of example, if the capacitor 42 has a capacitance of C=0 microfarads and the resistor 48 has a resistance of R=20K ohms, the RC time constant will be 200 milliseconds. This is substantially larger than the 1 millisecond pulse length produced by the photo diode array 24a-f. On the other hand, the RC time constant should not be so large as to prevent adequate DC current flow from the implanted body tissue into the capacitor 42 between pulses. According to design convention for RC circuits, a period of five time constants is required in order for an RC circuit capacitor to become fully charged. Note that the selected RC time constant of 200 milliseconds satisfies this requirement if the photodiode array 24a-f is pulsed at 1000 millisecond intervals, which is typical for pacemakers. Thus, there will be approximately five 200 millisecond time constants between every pulse. Stated another way, the RC time constant will be approximately one-fifth of the time interval between successive pulses.

FIG. 6A shows the square wave electrical pulses generated by the photo diode array 24a-f. FIG. 6B shows the actual electrical pulses delivered at the electrodes 44 and 46 due to the presence of the RC circuit provided by the capacitor 42 and the resistor 50. Note that the pulses of FIG. 6B are substantially square is shape due to the RC circuit's time constant being substantially larger than the input pulse width. FIG. 6B further shows that there is a small reverse potential between pulses that counteracts DC current build up in the stimulated tissue. Ideally, the area A₁ underneath each positive pulse of FIG. 6B will be equal to the area A₂ of negative potential that follows the positive pulse.

Turning now to FIG. 7, an opto-electric coupling device 60 according to an alternative embodiment is shown. The opto-electric coupling device includes the opto-electrical transducer 22. A high quality capacitor 62 delivers pulses from the opto-electrical transducer's photodiode array 24a-f to a tip electrode (shown schematically at 64). A return path is provided from a ring electrode (shown schematically at 66). The capacitor 62 forms part of a DC current discharge system 68 that also includes a resistor 70. The resistor 70 is connected across the capacitor 62 to discharge it between pulses. Exemplary values for the capacitor 62 and the resistor 70 are 10 microfarads and 20K ohms, respectively. This embodiment may be somewhat less preferable to the first embodiment of FIG. 5 because an additional path to the implanted tissue is provided through the resistor 70 and the tip electrode 64, which may permit a minimal amount of net DC current to flow into the implanted tissue. It is believed, however, that such minimal net DC current may well be so small as to not be of medical concern.

Accordingly, an opto-electric coupling device has been disclosed. While various embodiments of the invention have been shown and described, it should be apparent that many variations and alternative embodiments could be implemented in accordance with the invention. For example, although the opto-electric coupling devices 40 and 60 are shown in the context of a photonic pacemaker, they could be implemented in any opto-electric medical stimulation system wherein photonic signals are converted to electrical signals for use in stimulating body tissue. Such devices include, but are not limited to, defibrillators, neural stimulators, and other medical equipment designed to stimulate body tissue using electrical current. It is understood, therefore, that the invention is not to be in any way limited except in accordance with the spirit of the appended claims and their equivalents.

I claim:

1. An opto-electric coupling device for photonic pacemakers and other opto-electric medical stimulation equipment, comprising:
an opto-electrical transducer adapted to generate periodic electrical pulses across a pair of electrodes adapted for implantation in a body; and
a DC current discharge system adapted to counteract DC current flow caused by application of said electrical pulses to a body in which said electrodes are implanted.

2. An opto-electric coupling device in accordance with claim 1, wherein said opto-electrical transducer is adapted to deliver said electrical pulses from a first output thereof via a component of said DC current discharge system to one of said electrodes, and wherein a second output of said opto-electrical transducer is connected to a second one of said electrodes.

3. An opto-electric coupling device in accordance with claim 1, wherein said DC current discharge system is connected to discharge DC current from said body in which said electrodes are implanted.

4. An opto-electric coupling device in accordance with claim 1, wherein said DC current discharge system comprises a capacitor.

5. An opto-electric coupling device in accordance with claim 1, wherein said DC current discharge system comprises a capacitor and a resistor.

6. An opto-electric coupling device in accordance with claim 5, wherein said resistor is connected across a pair of outputs of said opto-electrical transducer.

7. An opto-electric coupling device in accordance with claim 5, wherein said resistor is connected across said capacitor.

8. An opto-electric coupling device in accordance with claim 1, wherein said DC current discharge system comprise an RC circuit.

9. An opto-electric coupling device in accordance with claim 1, wherein said RC circuit has a time constant in excess of a pulse width delivered by said photo diode array.

10. An opto-electric coupling device in accordance with claim 1, said RC circuit has a time constant that is approximately one-fifth of a time interval between successive pulses generated by said opto-electrical transducer.

11. A photonic pacemaker system, comprising:
an opto-electrical transducer adapted to generate periodic electrical pulses across a pair of electrodes;
a pair of electrodes for implantation in a body; and
a DC current discharge system adapted to counteract DC current flow caused by application of said electrical pulses to a body in which said electrodes are implanted.

12. An opto-electric medical stimulation system, comprising:
an opto-electrical transducer adapted to generate periodic electrical pulses across a pair of electrodes;
a pair of electrodes adapted for implantation in a body; and
a DC current discharge system adapted to counteract DC current flow caused by application of said electrical pulses to a body in which said electrodes are implanted.
